(19) United States
(12) Patent Application Publication
(10) Pub. No.: US 2015/0297772 A1
(43) Pub. Date: Oct. 22, 2015

(54) COMPOSITION FOR ODOR REMOVAL AND FRAGRANCE EMISSION COMPRISING COMPLEXES OF CUCURBITURIL AND FRAGRANCE

(71) Applicant: POSTECH ACADEMY-INDUSTRY FOUNDATION, Pohang-si, Gyeongsangbuk-do (KR)

(72) Inventors: Kimoon KIM, Pohang-si (KR); Young Ho KO, Pohang-si (KR); Su Hyun LEE, Pohang-si (KR); Selvapalam NARAYANAN, Pohang-si (KR); Don Wook LEE, Pohang-si (KR)

(73) Assignee: POSTECH ACADEMY-INDUSTRY FOUNDATION, Pohang-si, Gyeongsangbuk-do (KR)

(21) Appl. No.: 14/443,358

(22) PCT Filed: Nov. 15, 2013
(86) PCT No.: PCT/KR2013/010435

§ 371 Date: May 15, 2015

(30) Foreign Application Priority Data
Nov. 16, 2012 (KR) 10-2012-0130351
Mar. 19, 2013 (KR) 10-2013-0029322

(51) Int. Cl. A61L 9/01

(52) U.S. Cl. CPC A61L 9/01

(57) ABSTRACT

Disclosed herein is a composition for removing an odor molecule and releasing a fragrant molecule, the composition including a complex of cucurbituril and a fragrant molecule. The composition is characterized in that an odor molecule and a fragrant molecule alternately make a complex with cucurbituril, so the odor molecule makes a complex with cucurbituril, and, simultaneously, the fragrant molecule is released from the complex. The composition provides very efficient air cleaning effects because it can exhibit both an odor removing effect and a fragrant molecule releasing effect using only cucurbituril which is a single-kind macrocyclic molecule.

### TECHNICAL FIELD

The present invention relates to a composition for removing an odor and releasing a fragrant molecule, the composition including a complex of cucurbituril and a fragrant molecule. The composition is characterized in that an odor molecule and a fragrant molecule alternately makes a complex with cucurbituril, so the odor molecule makes a complex with cucurbituril, and, simultaneously, the fragrant molecule is released from the complex.

### BACKGROUND ART

Generally, an air freshener includes a capture material for removing an odor and a fragrant material for providing a fragrance. However, when a macrocyclic molecule having binding force to both odor molecule and fragrant molecule is used, two functions for removing an odor and providing a fragrance can be simultaneously realized by releasing a fragrant molecule from a complex of a fragrant molecule and a macrocyclic molecule while the complex is capturing an odor molecule. For this purpose, cucurbituril, having lately attracted considerable attention, can be used as a macrocyclic molecule. Cucurbit[6]uril is a macrocyclic compound composed of six monomers. Later, in 2000, K. Kim and others synthesized and separated cucurbit[n]uril [n] (n=5, 7, 8), as homologues of cucurbit[6]uril, as well as cucurbit[6]uril, by improving a conventional method of synthesizing cucurbit[6]uril, and they observed their respective structures using X-ray crystallogrphy (J. Am. Chem. Soc. 2000, 122, 540). Meanwhile, International Patent Application Publication No. WO 2000/68232 discloses cucurbit[n]uril which is represented by Chemical Formula below:

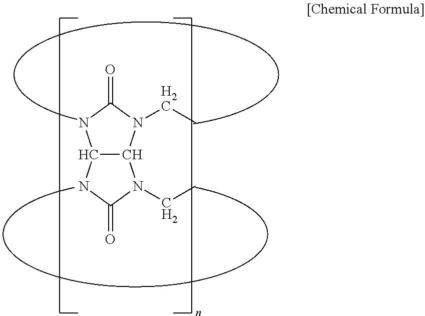

wherein n is an integer of 4 to 12.

Each of the cucurbituril homologues is a compound composed of glycoluril monomers having no substituent group (KR-A-10-2007-0073225).

It is proposed in the prior patent of the present inventors that cucurbituril can be used as a bad odor remover or the like because it has a structure which can make a complex with an organic compound. However, experiments that may support this possibility have never been carried out by the present inventors or other researchers. Thus, the present inventors experimentally found that, when cucurbituril, the cavity of which makes a complex with a fragrant molecule, makes a complex with an odor molecule, the odor molecule is exchanged with the fragrant molecule, and thus both a function of removing the odor molecule and a function of releasing the fragrant molecule can be realized, and the present inventors also found the optimal composition ratio of the complex and the optimal conditions required to exhibit functions. Based on these findings, the present invention was completed.

### DISCLOSURE

### Technical Problem

Accordingly, the present invention has been devised to solve the above-mentioned problems, and an object of the present invention is to provide a composition for removing an odor molecule and releasing a fragrant molecule, the composition including a complex of cucurbituril and a fragrant molecule. This composition is characterized in that an odor molecule makes a complex with the complex, and, simultaneously, a fragrant molecule is released from the complex.

However, an object to be accomplished by the present invention is not limited to the above-mentioned object, and other objects (not mentioned) will be clearly understood from the following descriptions by those skilled in the art.

### Technical Solution

In order to accomplish the above object, an aspect of the present invention provides a composition for removing an odor molecule and releasing a fragrant molecule, the composition including a complex of a fragrant molecule and a compound represented by Chemical Formula 1 below:

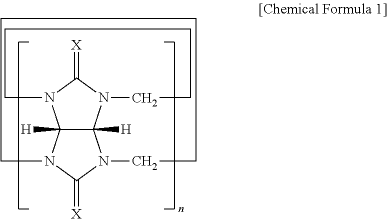

wherein n is an integer of 4 to 20, and X is O, S or NH,

wherein the composition includes 45 to 100 wt % of the compound of Chemical Formula 1 of n=7, and

wherein cucurbituril makes a complex with an odor molecule, and simultaneously a fragrant molecule is released from the complex.

The compound of Chemical Formula 1 is cucurbituril.

### Advantageous Effects

As described above, the composition of the present invention provides very efficient air cleaning effects because it can exhibit both an odor removing effect and a fragrant molecule releasing effect using only cucurbituril, which is a single-kind macrocyclic molecule.

### DESCRIPTION OF DRAWINGS

The above and other objects, features and advantages of the present invention will be more clearly understood from the following detailed description taken in conjunction with the accompanying drawings, in which:

FIG. 1 is a schematic view showing a complex of cucurbituril and a fragrant molecule, wherein an odor molecule makes a complex with cucurbituril and a fragrant molecule is released from cucurbituril;

FIG. 2 is a view showing the 1H-NMR results of menthol associated with cucurbit[6]uril, cucurbit[7]uril and cucurbit[8]uril;

FIG. 3 is a view showing the release of ethyl butyrate from a complex of cucurbituril and ethyl butyrate when diallydisulfide (odor molecule) is added to the complex;

FIG. 4 is a view showing the release of menthol from a complex of cucurbituril and menthol when diaminohexane (odor molecule) is added to the complex; and

FIG. 5 is a view showing the release of hexanal from a complex of cucurbituril and hexanal when diaminohexane (odor molecule) is added to the complex.

### EMBODIMENT

Hereinafter, embodiments of the present invention will be described in detail with reference to the attached drawings.

The present invention provides a composition for removing an odor molecule and releasing a fragrant molecule, the composition including a complex of a fragrant molecule and a compound represented by Chemical Formula 1 below,

wherein the composition includes 45 to 100 wt % of the compound of Chemical Formula 1 of n=7, and

cucurbituril makes a complex with an odor molecule, and a fragrant molecule is released from the complex.

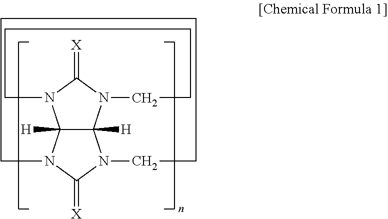

Here, n is an integer of 4 to 20, and X is O, S or NH.

The compound of Chemical Formula 1 is cucurbituril.

According to the experiment of the present inventors (refer to Example 1), since the cavity of cucurbit[7]uril, which is the compound of Chemical Formula 1 of n=7, is most easily associated with a fragrant material, cucurbit[7]uril is most advantageous in terms of releasing a fragrant molecule. In this case, it is preferred that the ratio of the amount of cucurbit[7]uril to the total amount of cucurbituril be high. However, in order to increase the amount of cucurbit[7]uril at the time of synthesizing cucurbit[7]uril, a process of purifying the synthesized cucurbit[7]uril (crude cucurbit[7]uril) is additionally required, thus decreasing economical efficiency. Therefore, the synthesized crude cucurbit[7]uril itself can be used to release a fragrant molecule and remove an odor molecule, and, in this case, the amount of cucurbit[7]uril in the crude cucurbit[7]uril may be 45% to 100% based on the total amount of cucurbituril. The composition may further include cucurbiturils of Chemical Formula 1 of n=4 to 6 and 8 to 20 in an amount of 0˜35%. Among the cucurbiturils of Chemical Formula 1 of n=4 to 6 and 8 to 20, cucurbit[6]uril is mostly used. Besides, the composition may further include cucurbiturils of Chemical Formula 1 of n=4, 5 and 8 to 20 in an amount of 0˜5%.

The crude cucurbit[7]uril is synthesized by a method including the following steps. However, the synthesis method thereof is not limited thereto.

1) Glycoluril 1.05 M (150 g) and paraformaldehyde 2.2 M (67 g) are mixed with a sufficient amount of HCl (225 mL), and this mixture is heated to 85˜90° C. for 18 hours to obtain a cucurbituril solution.

2) H₂O (3 L) is added to the cucurbituril solution, and then the solution is filtered to obtain a filtrate, and then the filtrate is mixed with methanol (2.4 L) to obtain a solid-containing solution.

3) The solid-containing solution is filtered by a filter paper to obtain a solid compound, and then the solid compound is washed with methanol (2 L) and dried to obtain crude cucurbit[7]uril.

According to an embodiment of the present invention, the complex of a compound of Chemical Formula 1 and a fragrant molecule is characterized in that the molar ratio of the compound to the fragrant molecule is 1:0.5˜1. Preferably, the molar ratio of the compound to the fragrant molecule may be 1:1. Cucurbituril has a cavity. As the number of fragrant molecules charged in the cavity increases, the binding ability of cucurbituril and fragrant molecules increases. Therefore, when a large number of fragrant molecules make a complex with cucurbituril, a large number of fragrant molecules are released, but characteristics of the complex are maintained constant regardless of the binding ability thereof, and the complex is more easily formed when the molar ratio of cucurbituril to the fragrant molecule is 1:0.5˜1. The composition ratio of the complex is determined according to the solubility of a fragrant material in water and the amount of volatilized fragrant material with respect to temperature.

According to another embodiment of the present invention, the fragrant molecule may be selected from the group consisting of ethyl butyrate, camphor, menthol, hexanal, eugenol, citronellol, linalool, patchoulol, santalol, phenyl ethyl alcohol, terpineol, vanillin, benzaldehyde, methyl salicylate, isoamyl acetate, d-limonene, L-limonene, α-pinene, 2-hydroxy-p-cymene, methyl acetophenone, and cis-jasmone. However, the kinds of the fragrant molecules are not limited thereto.

According to still another embodiment of the present invention, the composition may further include water. According to the experiment of the present inventors, the fragrant molecule releasing function of cucurbituril is maximized when it exists in the state of an aqueous solution compared to when it exists in the dry state.

A method of preparing a complex of cucurbituril and a fragrant molecule includes the steps of: (i) dissolving cucurbituril in an aqueous solution to obtain a mixed solution; and (ii) adding a fragrant molecule to the mixed solution.

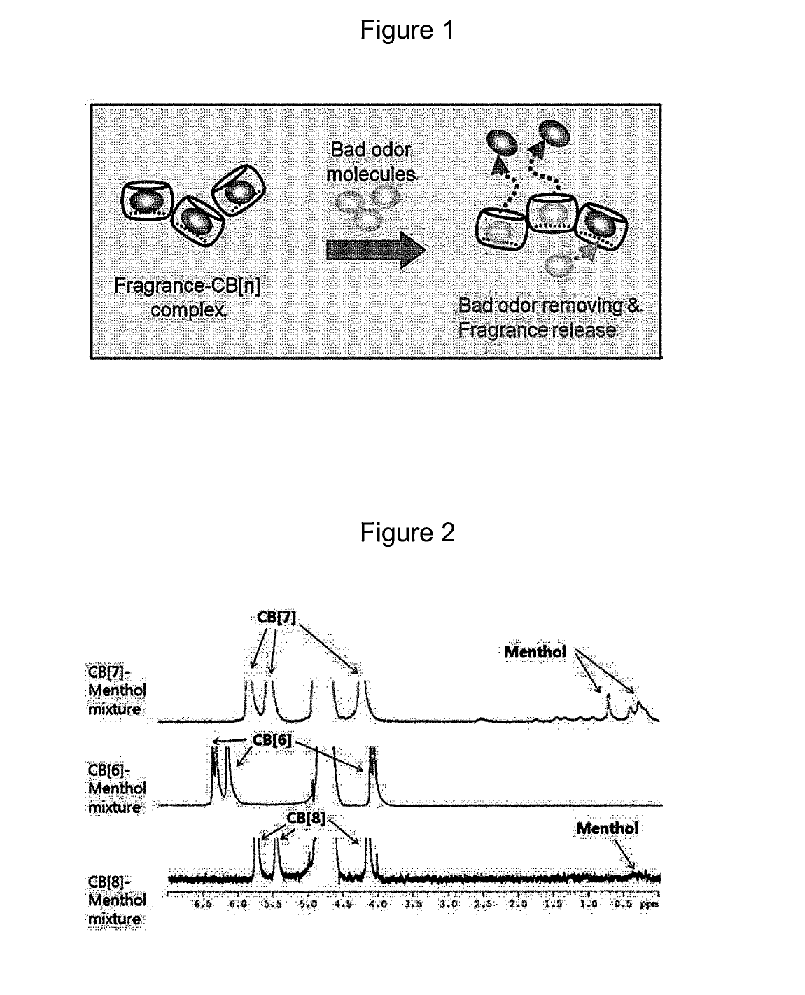

Cucurbituril can make a complex with various kinds of molecules by different bonding forces because it has a hydrophobic cavity provided therein and carbonyl group provided at both inlets thereof. Therefore, when a cucurbituril molecule makes a complex with a fragrant molecule to prepare a cucurbituril complex and then an odor molecule is added to the cucurbituril complex, the fragrant molecule is released from the cucurbituril-fragrant molecule complex, and, simultaneously, the odor molecule makes a complex with this complex instead of the fragrant molecule (refer to FIG. 1). Based on this principle, the present inventors made a complex which can remove odor from an odor source and simultaneously can release a fragrance.

Hereinafter, the present invention will be described in more detail with reference to the following Examples. However, these Examples are set forth to illustrate the present invention, and the scope of the present invention is not limited thereto.

### EXAMPLES

### Example 1

### Determining Cucurbituril Suitable for Preparing a Fragrant Molecule Complex

1-1: Determining Cucurbituril

There are various cucurbituril homologues and derivatives. The present inventors expected that cucurbit[7]uril, among the cucurbituril homologues and derivatives, would be most suitable for preparing a fragrant molecule complex. The reason for this is as follows.

First, cucurbit[7]uril can easily capture a fragrant molecule because it has a large cavity. In contrast, cucurbit[5]uril, cucurbit[6]uril cannot easily make a complex with a fragrant molecule because it has very small cavity. (* Comparison of sizes of cavities of cucurbituril homologues: cucurbit[8]uril>cucurbit[7]uril>cucurbit[6]uril>cucurbit[5]uril)

Second, cucurbit[7]uril is easily dissolved in water because it has high solubility in water. The solubility of cucurbit[6]uril or cucurbit[8]uril in water is less than 10⁻⁵ M because it has a symmetric molecule structure. In contrast, the solubility of cucurbit[7]uril in water is about 2˜3×10⁻² M, which is high.

Therefore, the present inventors carried out the following experiment in order to confirm the expected results.

A complex was prepared by mixing 100 mg of cucurbit[7]uril and 11.8 mg of menthol at a molar ratio of 1:1. 79.5 mg of cucurbit[6]uril and 118.3 mg of cucurbit[8]uril were respectively put into two mortars, and 11.8 mg of menthol was added to each of the mortars. 0.5 mL of distilled water was added to form a slurry, and then the slurry was stirred for 10 minutes using a pestle to prepare a complex. The complex was dried in an oven at 40 r for 6 hours. The dried complex was analyzed by 1H-NMR.

The 1H-NMR results thereof are shown in FIG. 2. As shown in FIG. 2, it can be ascertained that cucurbit[6]uril barely makes a complex with menthol, cucurbit[8]uril weakly makes a complex with menthol, and cucurbit[7]uril strongly makes a complex with menthol.

### Example 2

### Preparation of a Complex of Cucurbituril and a Fragrant Molecule (Ethyl Butyrate) and Evaluation of Function of the Complex

Cucurbit[7]uril was dissolved in distilled water to obtain a 2 mM cucurbit[7]uril aqueous solution. Ethyl butyrate (2 mM), which is a fragrant material, was added to the solution to obtain a complex (cucurbit[7]uril:ethyl butyrate=1:1). The bonding of diallyldisulfide with cucurbituril according to the addition of diallyldisulfide, which is an odor material, and the release of ethyl butyrate according to the addition of diallyldisulfide were evaluated comparing the 1H-NMR peak of the complex after the addition of diallyldisulfide with the 1H-NMR peak of the complex before the addition of diallyldisulfide.

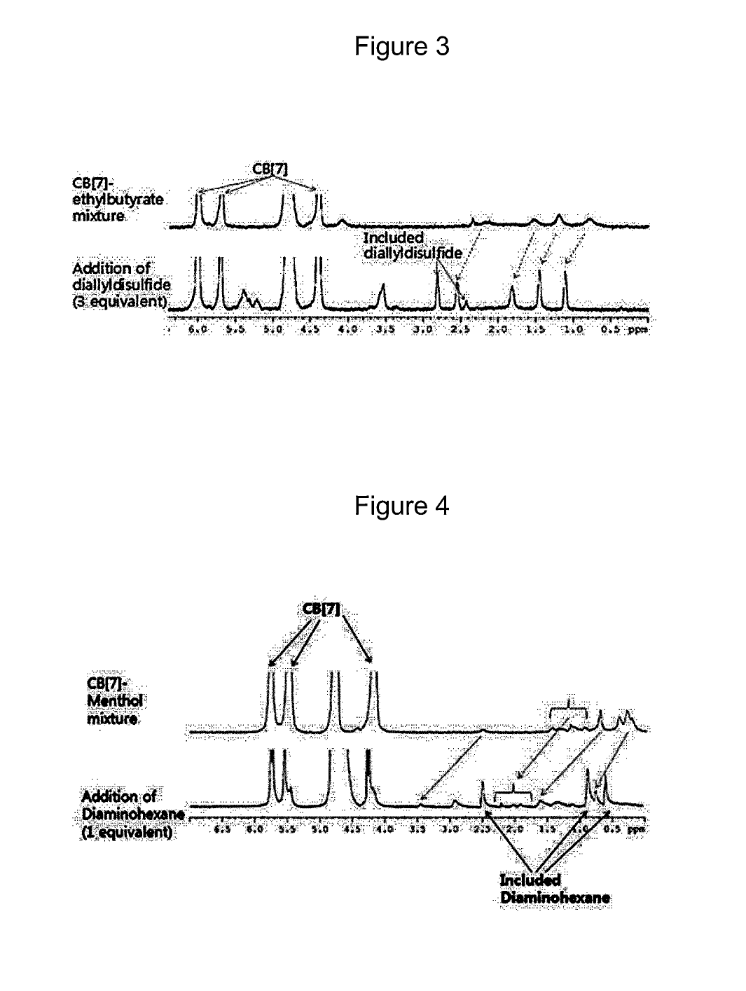

FIG. 3 is a view showing the release of ethyl butyrate from a complex of cucurbituril and ethyl butyrate when diallydisulfide (odor molecule) is added to the complex. From FIG. 3, it can be seen that ethyl butyrate associated with cucurbituril is released from the complex, and thus the release rate of ethyl butyrate from the complex increases compared to that of a conventional complex, and that, when diallydisulfide is added to the complex, diallydisulfide makes a complex with cucurbituril, and thus the release rate of ethyl butyrate from the complex is newly set.

### Example 3

### Preparation of a Complex of Cucurbituril and a Fragrant Molecule (Menthol) and Evaluation of Function of the Complex

Cucurbit[7]uril was dissolved in distilled water to obtain a 4 mM cucurbit[7]uril aqueous solution. Menthol (4 mM), which is a fragrant material, was added to the solution to obtain a complex (cucurbit[7]uril:menthol=1:1). The bonding of diaminohexane with cucurbituril according to the addition of diaminohexane, which is an odor material, and the release of menthol according to the addition of diaminohexane were evaluated comparing the 1H-NMR peak of the complex after the addition of diaminohexane with the 1H-NMR peak of the complex before the addition of diaminohexane.

FIG. 4 is a view showing the release of menthol from a complex of cucurbituril and menthol when diaminohexane (odor molecule) is added to the complex. From FIG. 4, it can be seen that menthol associated with cucurbituril is released from the complex, and then returns to its original position, and that, when diaminohexane is added to the complex, diaminohexane makes a complex with cucurbituril, and thus the release rate of menthol from the complex is newly set.

### Example 4

### Preparation of a Complex of Cucurbituril and a Fragrant Molecule (Hexanal) and Evaluation of Function of the Complex

Cucurbit[7]uril was dissolved in distilled water to obtain a 4 mM cucurbit[7]uril aqueous solution. Hexanal (4 mM), which is a fragrant material, was added to the solution to obtain a complex (cucurbit[7]uril:hexanal=1:1). The bonding of diaminohexane with cucurbituril according to the addition of diaminohexane, which is an odor material, and the release of hexanal according to the addition of diaminohexane were evaluated comparing the 1H-NMR peak of the complex after the addition of diaminohexane with the 1H-NMR peak of the complex before the addition of diaminohexane.

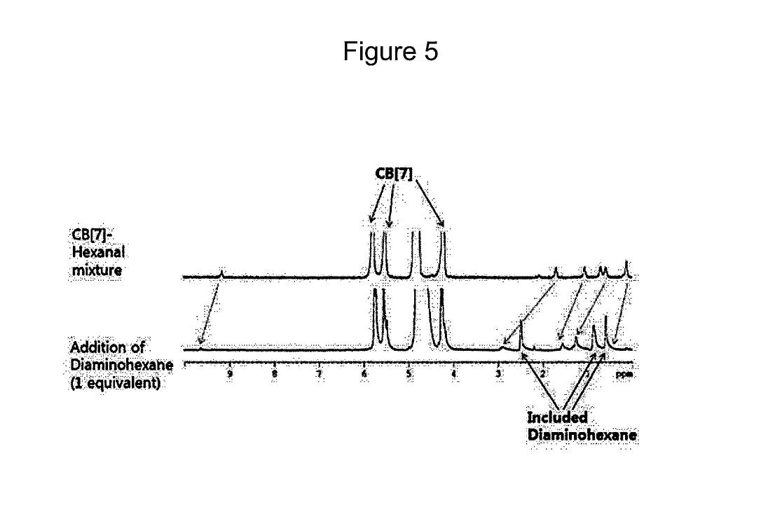

FIG. 5 is a view showing the release of hexanal from a complex of cucurbituril and hexanal when diaminohexane (odor molecule) is added to the complex. From FIG. 5, it can be seen that hexanal associated with cucurbituril is released from the complex, and then returns to its original position, and that, when diaminohexane is added to the complex, diaminohexane makes a complex with cucurbituril, and thus the release rate of hexanal from the complex is newly set.

Although the embodiments of the present invention have been disclosed for illustrative purposes, those skilled in the art will appreciate that various modifications, additions and substitutions are possible, without departing from the scope and spirit of the invention as disclosed in the accompanying claims.

### INDUSTRIAL APPLICABILITY

The composition is characterized in that an odor molecule and a fragrant molecule alternately make a complex with cucurbituril, so the odor molecule makes a complex with cucurbituril, and, simultaneously, the fragrant molecule is released from the complex. The composition provides very efficient air cleaning effects because it can exhibit both an odor removing effect and a fragrant molecule releasing effect using only cucurbituril which is a single-kind macrocyclic molecule.

1. A composition for removing an odor molecule and releasing a fragrant molecule, the composition comprising a first complex of a fragrant molecule and a compound represented by Chemical Formula 1 below:
[Chemical Formula 1] 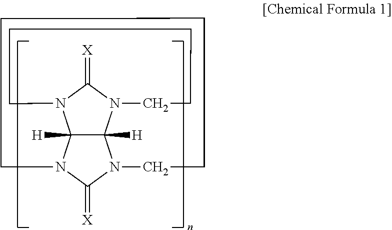
wherein n is an integer of 4 to 20, and X is O, S or NH,
wherein the composition includes 45 to 100 wt % of the compound of Chemical Formula 1 of n=7, and
wherein the first complex is configured to release the fragrant molecule while forming a second complex with a foreign odor molecule.

2. The composition of claim 1, wherein the first complex of the compound of Chemical Formula 1 and the fragrant molecule has a molar ratio of the compound to the fragrant molecule ranging from to 1.

3. The composition of claim 1, wherein the fragrant molecule is selected from the group consisting of ethyl butyrate, camphor, menthol, hexanal, eugenol, citronellol, linalool, patchoulol, santalol, phenyl ethyl alcohol, terpineol, vanillin, benzaldehyde, methyl salicylate, isoamyl acetate, d-limonene, L-limonene, α-pinene, 2-hydroxy-p-cymene, methyl acetophenone, and cis-jasmone.

4. The composition of claim 1, further comprising water.

5. A method of air freshening comprising placing the composition of claim 1 in an environment having odor molecules and releasing a fragrant molecule, wherein the first complex releases the fragrant molecule and forms a second complex with one of the odor molecules from the environment.
